(19) United States
(12) Patent Application Publication
(10) Pub. No.: US 2020/0234824 A1
(43) Pub. Date: Jul. 23, 2020

(54) WOUND TREATMENT RECOMMENDATION SYSTEM AND WOUND TREATMENT RECOMMENDATION METHOD

(71) Applicant: Industrial Technology Research Institute, Hsinchu (TW)

(72) Inventors: Yue-Min JIANG, New Taipei City (TW); Ho-Hsin LEE, Hsinchu City (TW); I-Ju YEH, Hsinchu City (TW); Jian-Ren CHEN, Hsinchu City (TW); Su-Chen HUANG, Taoyuan City (TW)

(73) Assignee: Industrial Technology Research Institute, Hsinchu (TW)

(21) Appl. No.: 16/286,110

(22) Filed: Feb. 26, 2019

(30) Foreign Application Priority Data
Jan. 22, 2019 (TW) 108102351

(51) Int. Cl. G16H 50/20; G16H 50/70; G16H 10/20

(52) U.S. Cl. CPC G16H 50/20; G16H 10/20; G16H 50/70

(57) ABSTRACT

A wound treatment recommendation method includes: storing a database; receiving a wound characterization record; generating a current data sequence according to the wound observation data in the wound characterization record; calculating similarity parameters of the current data sequence to each of case data sequences; regarding one of the similarity parameters with a highest restoration parameters and under a similarity threshold as the best case; selecting the questions used in the best case, excluding a plurality of existed questions in a questionnaire record, so as to establish a suggested questionnaire, and obtaining a plurality of answers of the suggested questionnaire; including all multiple reference cases within a specific range of values in each of the answers; and selecting multiple treatment cases including a nice recovery record from a plurality of treatment groups as at least one recommended treatment.

### CROSS REFERENCE TO RELATED APPLICATIONS

This application claims priority of Taiwan Patent Application No. 108102351, filed on 2019 Jan. 22, the entirety of which is incorporated by reference herein.

### BACKGROUND OF THE INVENTION

### Field of the Invention

The present disclosure relates to a recommendation system and a recommendation method, and in particular, to a wound treatment recommendation system and a wound treatment recommendation method suitable for wound treatment.

### Description of the Related Art

Wound care is currently performed by nurses. Nurses only judge according to the current condition of the wound, such as the cleaning and disinfection of basic wound care. However, under simple care, it is easy for a wound to stay in an inflammatory or proliferative phase, delaying the recovery of the wound. Alternatively, a wound is often unable to heal due to misjudgment, which may increase the risk of infection or necrosis, resulting in sepsis or amputation.

In addition, because the guidelines are complex and multi-faceted, it is difficult to judge completely. The general dogmatic guidelines tend to have priority or conflict in the face of complex situations, which makes it more difficult for the nurse to care for the wounds of the patients. Also, although there are only a dozen types of materials and dressings, there are many kinds of products, which often cause problems in the selection of products. It is often necessary to take advantage of the experience of professionals, and more observations and suggestions are needed.

Therefore, how to provide a wound treatment recommendation system and a recommendation method for wound disposal has become one of the problems to be solved in the field.

### BRIEF SUMMARY OF THE INVENTION

In accordance with one feature of the present invention, the present disclosure provides a wound treatment recommendation system. The wound treatment recommendation system comprises a storage device, a receiving device and a processor. The storage device stores a database. The database is used to record a plurality of reference cases. Each of the reference cases comprises a plurality of case data sequences and a plurality of treatment cases. The receiving device obtains a wound characterization record. The processor generates a current data sequence according to the wound observation data in the wound characterization record. The processor calculates a plurality of similarity parameters for the current data sequence to each of the case data sequences. The processor regards one of the restoration parameters with a highest restoration parameters and under a similarity threshold as the best case. The processor selects a plurality of questions used in the best case, excluding a plurality of existed questions in a questionnaire record, so as to establish a suggested questionnaire. The processor obtains a plurality of answers of the suggested questionnaire, includes all the reference cases within a specific range of values in each of the answers, selects the treatment cases comprising a nice recovery record from a plurality of treatment groups as the recommended treatment, and displays the recommended treatment on a display. The best case is the reference case with a highest proportion of wound area reduction per unit of time.

In accordance with one feature of the present invention, the present disclosure provides a wound treatment recommendation method. The wound treatment recommendation method comprises: storing a database; receiving the wound characterization record; generating the current data sequence according to wound observation data in the wound characterization record; calculating a plurality of similarity parameters of the current data sequence and each to each of the case data sequences; regarding one of the the similarity parameters with a highest restoration parameters and under a the similarity threshold as the best case; selecting the questions used in the best case, excluding a plurality of existed questions in the questionnaire record, so as to establish a suggested questionnaire, and obtaining a plurality of answers toof the suggested questionnaire; including all of the reference cases within a specific range of values in each of the answers, and selecting a plurality of treatment cases comprising a nice recovery record from a plurality of treatment groups as at least one recommended treatment; and displaying the at least one recommended treatment on a display. Each of the reference cases comprises case data sequences and treatment cases. The best case is the reference case with a highest proportion of wound area reduction per unit of time. The database is used to record the reference cases.

Based on the above, the wound treatment recommendation system and the wound treatment recommendation method can obtain the wound characterization record, and compare the wound characterization record with the case data sequence of each reference case to select the reference closest to the current wound. The case is accompanied by a suggested questionnaire to further obtain the answer to confirm the condition of the wound. All the reference cases within a certain range of values that differ from each answer result are included in a treatment group. The proposed treatment cases containing nice recovery records are selected from these treatment groups as recommended treatments. Furthermore, the treatments that have already been performed by a nurse or caregiver are filtered out from these proposed treatment cases, thereby allowing for a more streamlined treatment and providing better advice to the nurse or caregiver.

### BRIEF DESCRIPTION OF THE DRAWINGS

FIG. 1 is a block diagram of a wound treatment recommendation system in accordance with one embodiment of the present disclosure.

FIG. 2 is a flowchart of a wound treatment recommendation method in accordance with one embodiment of the present disclosure.

FIGS. 3A-3C are schematic diagrams of wound observation data in accordance with one embodiment of the present disclosure.

FIG. 4 is a schematic diagram of a suggested questionnaire generation method in accordance with one embodiment of the present disclosure.

FIG. 5 is a schematic diagram of a treatment group construction method in accordance with one embodiment of the present disclosure.

FIG. 6 is a schematic diagram of a recommended treatment selection method in accordance with one embodiment of the present disclosure.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present invention will be described with respect to particular embodiments and with reference to certain drawings, but the invention is not limited thereto and is only limited by the claims. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

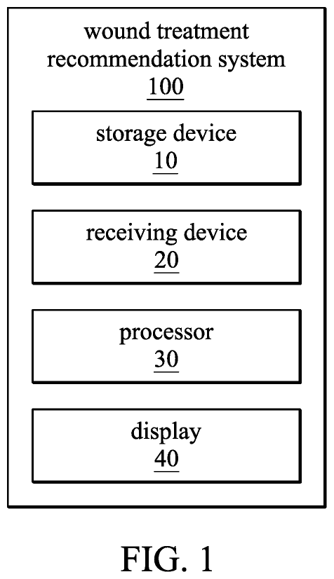

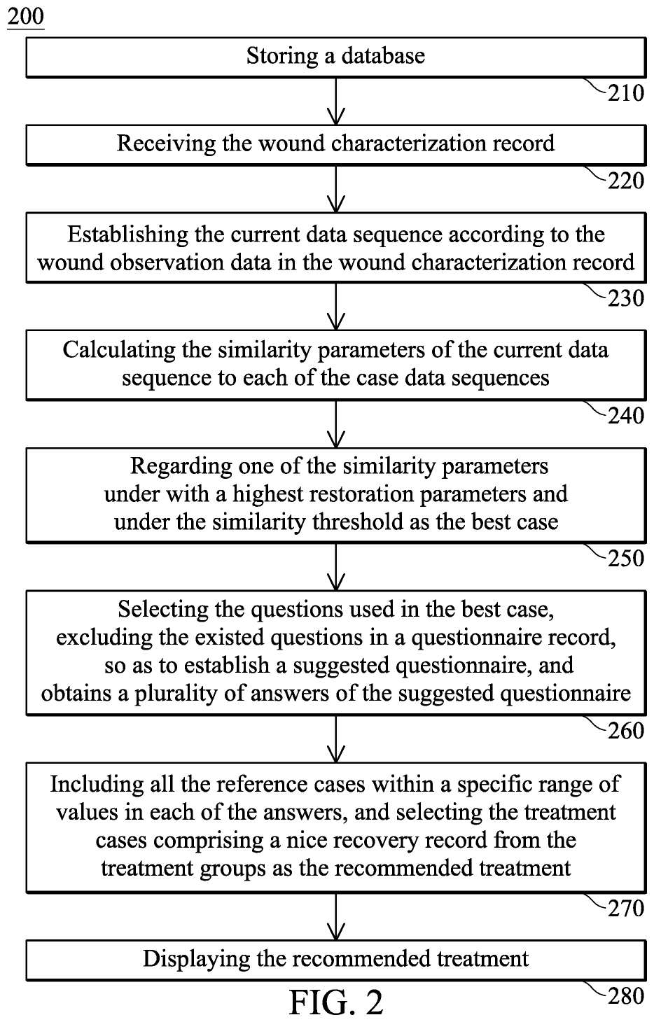

Please refer to FIGS. 1-2. FIG. 1 is a block diagram of a wound treatment recommendation system 100 in accordance with one embodiment of the present disclosure. FIG. 2 is a flowchart of a wound treatment recommendation method 200 in accordance with one embodiment of the present disclosure. The wound treatment recommendation system 100 comprises a storage device 10, a receiving device 20, a processor 30 and a display 40.

In one embodiment, the storage device 10 can be implemented by a read-only memory, a flash memory, a floppy disk, a hard disk, an optical disk, a flash disk, a magnetic tape, a database accessible via a network, or a storage medium that can be easily conceived by those of ordinary skill in the art and has the same function.

In one embodiment, the receiving device 20 can be a camera or any device that can obtain or receive information about the wound or the injured part (for example, a keyboard). In one embodiment, the camera captures the image of the wound or the injured part and transmits the image to the processor 30 for performing image analysis.

In one embodiment, the processor 30 can be implemented by, for example, a microcontroller, a microprocessor, a digital signal processor, an application specific integrated circuit (ASIC), or a logic circuit.

The procedure of the wound treatment recommendation method 200 is described as follows.

In step 210, the storage device 10 stores a database. The database is used to record the reference cases. Each reference case comprises a plurality of case data sequences and a plurality of treatment cases.

In one embodiment, the values corresponding to a measuring wound time in the case data sequences include a wound length, a wound width, a wound depth, a degree of wound fluid, and a skin temperature. For example, the case data sequences can express the healing day of wound, the wound length, the wound width, the wound depth, the degree of wound fluid, and the skin temperature as “day 01, {2.0, 3.0, 0.5, 50, 32, . . . }” and “day 07, {3.0, 2.0, 0.4, 25, 31, . . . }”. It means that when the number of days of the wound is the first day (for example, it is presented as “day 01”), the wound length is 2.0, the wound width is 3.0, the wound depth is 0.5, the degree of wound fluid is 50, and the skin temperature is 32. When the number of days of the wound is 7-th day (for example, it is presented as “day 07”), the wound length is 3.0, the wound width is 2.0, the wound depth is 0.4, the degree of wound fluid is 25, and the skin temperature is 31. The units of these values can be defined by the user.

With the case data sequences, it is easy to compare the degree of wound recovery at different times of measurement, and perform the treatment cases according to the wound condition by the professional advanced practice nurse. The treatment cases can be, for example, “turning over every two hours”, “applying disinfection and aseptic technique to dressing wounds”, “maintaining the cleanliness of the sheets”, etc. In one embodiment, the treatment cases include such case data sequences and their corresponding case treatment information. The processor 30 stores the reference cases to the storage device 10 for providing follow-up steps for reference.

In step 220, the receiving device 20 receives the wound characterization record. In one embodiment, the wound characterization record includes wound area variation, wound color, wound location, wound depth, wound area, or wound shape.

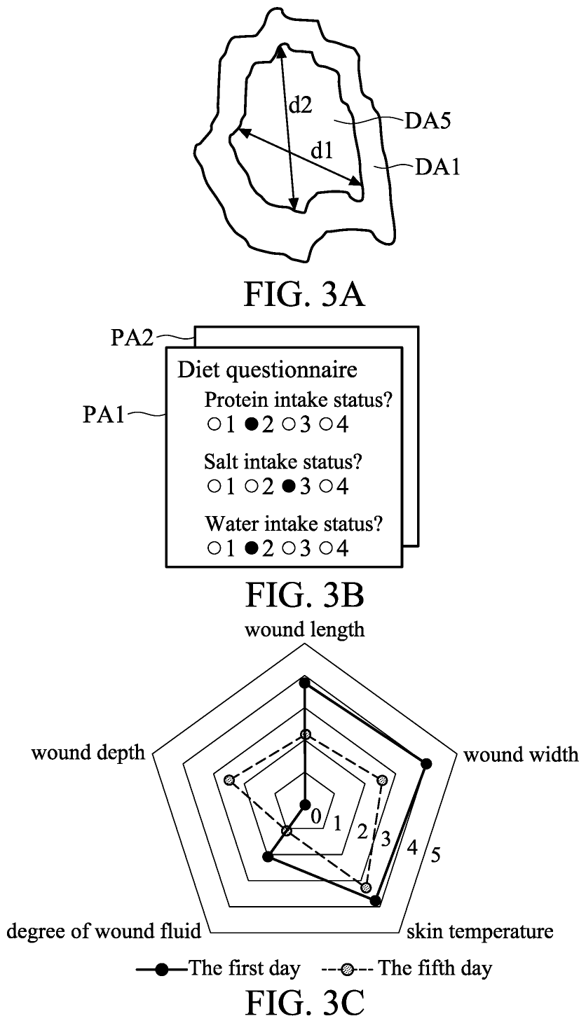

Please refer to FIGS. 3A-3C. FIGS. 3A-3C are schematic diagrams of wound observation data in accordance with one embodiment of the present disclosure. In one embodiment, the wound observation data includes measurement-type information (shown as FIG. 3A) or evaluation-type information (shown as FIG. 3B).

As shown in FIG. 3A, the measurement-type information can be obtained by analyzing the image through the processor 30. For example, an image is transmitted to the processor 30 after the camera takes the image of the wound or the injured part. The processor 30 performs the image analysis according to the color distribution of the image, coordinate positioning, or other known methods. For example, by obtaining the wound image DA1 on the first day and the wound image DA5 on the fifth day, the wound-related data can be further analyzed. For example, the wound width d1 and the wound length d2 are analyzed according to the wound image DA5 on the fifth day, thereby obtaining the wound observation data.

As shown in FIG. 3B, evaluation-type information can be obtained, for example, from an evaluation questionnaire. By having nurse, caregiver, or patient fill out multiple evaluation questionnaires (e.g., diet questionnaire PA1, life questionnaire PA2, etc.), each evaluation questionnaire contains multiple evaluation questions and their corresponding degree options (for example, it is provided 1 to 5 degrees), which allows a nurse, caregiver, or patient to select the corresponding degree option for the evaluation problem based on the current state of the wound, thereby obtaining wound observation data.

In step 230, the processor 30 establishes the current data sequence according to the wound observation data in the wound characterization record. In one embodiment, the current data sequence can be represented by a mathematical formula or a series of representations, which can be represented in a manner similar to the case data sequences.

In one embodiment, the processor 30 further establishes the current data sequence by normalizing the wound observation data. Since the normalization calculation is a known mathematical calculation method, it will not be further described here. In one embodiment, the processor 30 can convert the raw wound observation data to a specific range (for example, 0 to 5) by a known mathematical function.

In one embodiment, when the wound observation data is normalized, the data in the current data sequence established by the wound observation data can be presented in the manner shown in FIG. 3C.

In the step 240, the processor 30 calculates the similarity parameters of the current data sequence to each of the case data sequences.

In one embodiment, the similarity parameters can refer to the mathematical distance difference between the current data sequence and each case data sequence. For example, the current data sequence is “day 07, {2.0, 3.0, 0.5, 50, 32, . . . }”, the case data sequence A is “day 07, {2.0, 3.0, 0.5, 48, 31, . . . }”, the case data sequence B is “day 07, {10.0, 15.0, 0.9, 46, 31, . . . }”, and the current data sequence can be subtracted from each value in the case data sequence A to obtain a plurality of difference values, and then after adding the difference values, the result of the square root of the added difference values is the mathematical distance difference between the current data sequence and the case data sequence A. Similarly, the current data sequence is subtracted from each value in the case data sequence B to obtain a plurality of difference values, and then after adding the difference values, the result of the square root of the added difference values is the mathematical distance difference between the current data sequence and the case data sequence A. In this example, the mathematical distance difference between the current data sequence and the case data sequence A is smaller than the mathematical distance difference between the current data sequence and the case data sequence B. Therefore, the current data sequence has a higher similarity with the case data sequence A.

However, the calculation method of the similarity parameters is not limited thereto, and the mathematical calculation method which can be used to calculate the similarity between the current data sequence and each case data sequence can be applied. In addition, the similarity parameters are not limited thereto, and the similarity parameters may refer to an area difference between the current data sequence and each case data sequence, a wound fluid difference, a temperature difference, and the like.

In step 250, the processor 30 regards one of the similarity parameters with a highest restoration parameters and under the similarity threshold as the best case.

For example, if the similarity threshold is 5 and the restoration parameters (for example, 40%, 30%, and 50%) are lower than the similarity threshold, these restoration parameters correspond to the respective similarity parameters (for example, similarity parameters of 1, 0.7, and 2), the reference case that corresponds to the highest recovery parameter (i.e., a recovery parameter of 50%) is considered to be the best case. The reference case contains a recovery parameter, which can be the recovery state recorded by the advanced practice nurse when the wound was previously treated, and the recovery state is numerically described. Thereby, the processor 30 can select a reference case that is similar to the current data sequence and that has a good prognosis.

In one embodiment, the best case is the reference case with the highest proportion of wound area reduction per unit of time. In one embodiment, the recovery parameter can be obtained from numerically recovery state. For example, among the similarity parameters that are lower than a similarity threshold value of 5, the similarity parameters correspond to reference case A and reference case B. In reference case A, the wound area is reduced by 70% per unit of time. In reference case B, the wound area is reduced by 90% per unit of time. Therefore, reference case B is selected as the best case.

In step 260, the processor 30 selects the questions used in the best case, excluding the existed questions in a questionnaire record, so as to establish a suggested questionnaire, and obtains a plurality of answers of the suggested questionnaire. For example, some questions that the nurse has asked the patient recorded in the questionnaire. When these questions that the nurse has asked the patient are included in the questions in the questionnaire corresponding to the best case, these questions that the nurse has asked the patient will be deleted from the questionnaire. It can prevent the nurse from asking the same questions again.

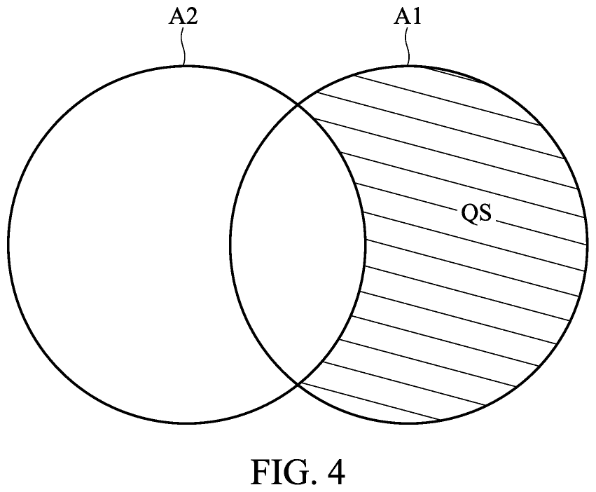

In one embodiment, please refer to FIG. 4. FIG. 4 is a schematic diagram of a suggested questionnaire generation method in accordance with one embodiment of the present disclosure. For example, In the process of dealing with the above-mentioned best case, the advanced practice nurse has asked the patient or the nurse, and these questions are regarded as the question set A1. Question set A1 is included in the data of the best case and is also recorded in the database in advance. Therefore, when the nurse finds the best case corresponding to the current wound by the wound treatment system 100, the question set A1 can be obtained, and after the question set A2 in the questionnaire record (question set A2 represents the questions that the nurse has already asked) is excluded, question set QS (as shown at the slash) is obtained. The processor 30 establishes the question set QS as a suggested questionnaire and displays it on the display 40 for the caregiver, patient or caregiver to answer.

In one embodiment, the processor 30 is further configured to generate a qualitative questionnaire according to the questions and the corresponding answers to the questions.

In step 270, the processor 30 includes all the reference cases within a specific range of values in each of the answers, and selects the treatment cases comprising a nice recovery record from the treatment groups as the recommended treatment.

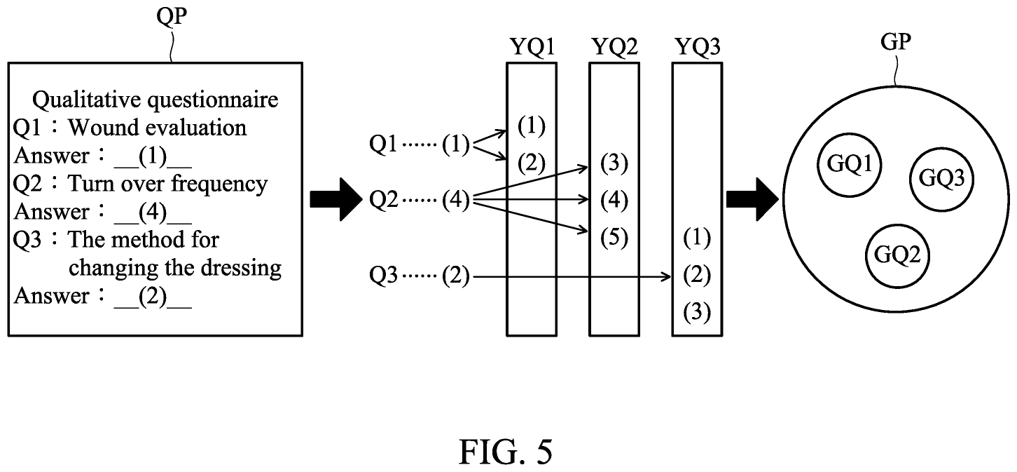

In one embodiment, please refer to FIG. 5. FIG. 5 is a schematic diagram of a treatment group construction method in accordance with one embodiment of the present disclosure. In FIG. 5, after the processor 30 obtaining the qualitative questionnaire QP, the processor 30 expands the answer result corresponding to each of the questions Q1 to Q3 in the qualitative questionnaire QP (according to the definition of the answer is extended for a specific range +1 to −1), and extracts the extension answer. The reference cases corresponding to the results of the subsequent answers are included in the respective treatment groups GQ1-GQ3.

More specifically, in this example, in the case where +1 to −1 is defined as the specific range for the result of the answer to expand. When the result of the question Q1 is option 1, the processor 30 expands the option to 1 to 2. The reference cases corresponding to the expanded answer results (1 and 2) are added to the treatment group GQ1. In other words, in all the reference case sets YQ1 that answered question Q1, the result of the answer is the reference case corresponding to options 1 and 2, which is included in the treatment group GQ1. When the result of the question Q2 is option 4, the processor 30 expands the option to 3 to 5 (taking the option of +1 of option 4 and the option of −1 of option 4). The reference cases corresponding to the expanded answer results (3 to 5) are added to the treatment group GQ2. In other words, in all the reference case sets YQ2 that answered question Q2, the result of the answer is the reference case corresponding to options 3 to 5, which is included in the treatment group GQ1. When the result of the question Q3 is option 2, the processor 30 expands the option to 1 to 3 (taking the option of +1 of option 2 and the option of −1 of option 2). The reference cases corresponding to the expanded answer results (1 to 3) are added to the treatment group GQ3. In other words, in all the reference case sets YQ3 that answered question Q3, the result of the answer is the reference case corresponding to options 1 to 3, which is included in the treatment group GQ3.

Therefore, when the result of the answer is slightly inaccurate with the actual situation, the reference case corresponding to the expanded answer result is included in the treatment group GQ1-GQ3. The treatment group GQ1-GQ3 is called the prescription question bank GP, which makes the probability that the actual situation is included in the prescription question bank GP becomes higher. For example, the actual body temperature is 37 degrees. Because of the human error in the measurement, the measurement of the body temperature is 38 degrees, and the temperature is expanded (+1 to −1 is defined as the specific range for the result of the answer to expand) as 36 to 37 degrees. The reference case corresponding to the correct actual body temperature of 37 degrees is included in the treatment group, which greatly improves the accuracy of the answer result.

In one embodiment, the processor 30 selects the reference cases comprising the nice recovery record (e.g., the recovery parameter is higher than a recovery parameter threshold) from the treatment groups, obtains candidate treatments corresponding to the reference cases, and takes the difference between the candidate treatments and the executed treatment, so as to select the recommended treatment. In this way, it is possible to filter out treatments that the nurse has already performed on the wound.

In one embodiment, the processor 30 obtains all the treatment cases in each of the treatment groups GQ1-GQ3 that are above a frequency threshold, without the executed treatment cases, as the recommended treatment.

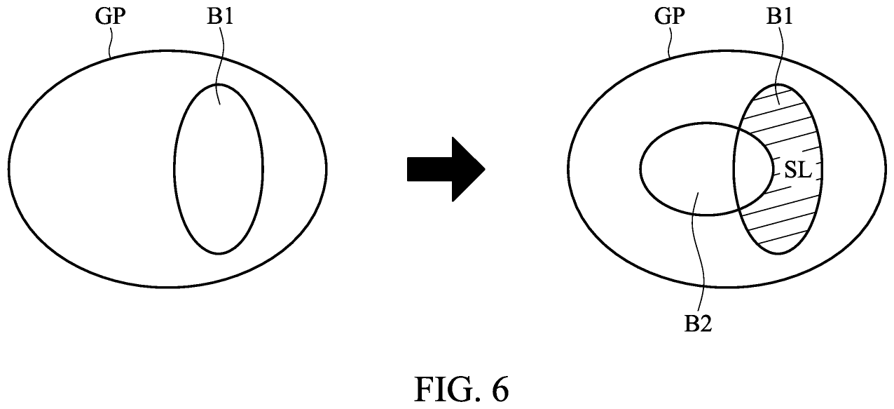

In one embodiment, please refer to FIG. 6. FIG. 6 is a schematic diagram of a recommended treatment selection method in accordance with one embodiment of the present disclosure. In FIG. 6, the processor 30 selects a treatment higher than a frequency threshold (for example, 70%) from each of the treatment groups GQ1-GQ3, and incorporates the treatment into the high frequency treatment area B1. For example, each of the treatment groups GQ1-GQ3 includes a treatment of “turning over every two hours”. The treatment of “turning over every two hours” occurs at a frequency of 100% for each treatment group GQ1-GQ3, which is greater than the frequency threshold (for example, 70%), the treatment of “turning over every 2 hours” is included in the high frequency treatment area B1 in the prescription question bank GP.

In one embodiment, the high frequency treatment area B1 includes multiple treatments. The processor 30 removes at least one case B2 of the executed treatment from the treatment of the high frequency treatment areas B1 to obtain the recommended treatment(s) SL. Therefore, in this example, the wound treatment recommendation system 100 can provide a high-frequency and widely-accepted treatment that has not been performed (which may be part that the nurse should pay attention, but ignore) according to the current wound state to the nurse.

In step 280, the recommended treatment is displayed on a display.

Based on the above, the wound treatment recommendation system and the wound treatment recommendation method can obtain the wound characterization record, and compare the wound characterization record with the case data sequence of each reference case to select the reference closest to the current wound. The case is accompanied by a suggested questionnaire to further obtain the answer to confirm the condition of the wound. All the reference cases within a certain range of values that differ from each answer result are included in a treatment group. The proposed treatment cases containing nice recovery records are selected from these treatment groups as recommended treatments. Furthermore, the treatments that have been performed by the nurse or caregiver are filtered out from these proposed treatment cases, thereby allowing for a more streamlined treatment and providing better advice to the nurse or caregiver.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such a feature may be combined with one or more other features of the other implementations as may be desired and advantageous for any given or particular application.

What is claimed is:

1. A wound treatment recommendation system, comprising: a storage device, configured to store a database; wherein the database is used to record a plurality of reference cases, and each of the reference cases comprises a plurality of case data sequences and a plurality of treatment cases;
a receiving device, configured to obtain a wound characterization record; and
a processor, configured to:
generate a current data sequence according to a plurality of wound observation data in the wound characterization record;
calculate a plurality of similarity parameters for the current data sequence to each of the case data sequences;
regard one of the similarity parameters with a highest restoration parameters and under a similarity threshold as a best case;
select a plurality of questions used in the best case, excluding existed questions in a questionnaire record, so as to establish a suggested questionnaire, and obtain a plurality of answers of the suggested questionnaire;
include all the reference cases within a specific range of values in each of the answers, and select the treatment cases comprising a nice recovery record from a plurality of treatment groups as at least one recommended treatment; and
display the at least one recommended treatment on a display;
wherein the best case is the reference cases with a highest proportion of wound area reduction per unit of time.

2. The wound treatment recommendation system of claim 1, wherein the processor is further configured to establish the current data sequence by normalizing the wound observation data.

3. The wound treatment recommendation system of claim 1, wherein the processor is further configured to generate a qualitative questionnaire according to the questions and corresponding answers to the questions.

4. The wound treatment recommendation system of claim 1, wherein the processor is further configured to select the reference cases comprising the nice recovery record from the treatment groups, and obtain a plurality of candidate treatments corresponding to the reference cases, and take a difference between the candidate treatments and at least one executed treatments to select the at least one recommended treatment.

5. The wound treatment recommendation system of claim 1, wherein the processor is further configured to obtain all the treatment cases in each of the treatment groups that are above a frequency threshold, and not at least one executed treatment cases, as the at least one recommended treatment.

6. The wound treatment recommendation system of claim 1, wherein the wound observation data comprises a measurement-type information or an evaluation-type information, and the wound characterization record comprises a wound area variation, a wound color, a wound location, a wound depth, a wound area, or a wound shape.

7. The wound treatment recommendation system of claim 1, wherein values corresponding to a measuring wound time in the case data sequences comprises a wound length, a wound width, a wound depth, a degree of wound fluid, and a skin temperature.

8. A wound treatment recommendation method, comprising:
storing a database; wherein the database is used to record a plurality of reference cases, and each of the reference cases comprises a plurality of case data sequences and a plurality of treatment cases;
receiving a wound characterization record;
generating a current data sequence according to a plurality of wound observation data in the wound characterization record;
calculating a plurality of similarity parameters of the current data sequence to each of the case data sequences;
regarding one of the similarity parameters with a highest restoration parameters and under a similarity threshold as the best case;
selecting the questions used in the best case, excluding a plurality of existed questions in a questionnaire record, so as to establish a suggested questionnaire, and obtaining a plurality of answers of the suggested questionnaire;
including all the reference cases within a specific range of values in each of the answers, and selecting the treatment cases comprising a nice recovery record from a plurality of treatment groups as at least one recommended treatment; and
displaying the at least one recommended treatment on a display;
wherein the best case is the reference case with a highest proportion of wound area reduction per unit of time.

9. The wound treatment recommendation method of claim 8, further comprising:
establishing the current data sequence by normalizing the wound observation data.

10. The wound treatment recommendation method of claim 8, further comprising:
generating a qualitative questionnaire according to the questions and corresponding answers to the questions.

11. The wound treatment recommendation method of claim 8, further comprising:
selecting the reference cases comprising the nice recovery record from the treatment groups, and obtaining a plurality of candidate treatments corresponding to the reference cases, and taking a difference between the candidate treatments and the at least one executed treatment to select the at least one recommended treatment.

12. The wound treatment recommendation method of claim 8, further comprising: obtaining all the treatment cases in each of the treatment groups that are above a frequency threshold, and not at least one executed treatment cases, as the at least one recommended treatment.

13. The wound treatment recommendation method of claim 8, wherein the wound observation data comprises a measurement-type information or an evaluation-type information, and the wound characterization record comprises a wound area variation, a wound color, a wound location, a wound depth, a wound area, or a wound shape.

14. The wound treatment recommendation method of claim 8, wherein the values corresponding to a measuring wound time in the case data sequences comprises a wound length, a wound width, a wound depth, a degree of wound fluid, and a skin temperature.
